(19) United States
(12) Patent Application Publication
(10) Pub. No.: US 2021/0056690 A1
(43) Pub. Date: Feb. 25, 2021

(54) MEDICAL INFORMATION PROCESSING APPARATUS AND COMPUTER READABLE STORAGE MEDIUM

(71) Applicant: Konica Minolta, Inc., Tokyo (JP)

(72) Inventors: Hitoshi FUTAMURA, Tokyo (JP); Satoshi KASAI, Tokyo (JP); Shinsuke KATSUHARA, Tokyo (JP)

(21) Appl. No.: 16/993,339

(22) Filed: Aug. 14, 2020

(30) Foreign Application Priority Data
Aug. 20, 2019 (JP) 2019-150116

(51) Int. Cl. G06T 7/00; G06T 7/70; G06K 9/32; G06N 20/00; G16H 30/20

(52) U.S. Cl. CPC G06T 7/0012; G06T 7/70; G06K 9/3233; A61B 5/055; G16H 30/20; G06T 2207/30096; G06N 20/00

(57) ABSTRACT

A medical information processing apparatus includes a hardware processor. The hardware processor determines, based on a predetermined condition, priority degrees of lesion-detected regions of multiple types of lesions detected in a medical image. Further, the hardware processor makes display forms of the lesion-detected regions detected in the medical image differ according to the determined priority degrees.

### CROSS-REFERENCE TO RELATED APPLICATIONS

The entire disclosure of Japanese Patent Application No. 2019-150116 filed on Aug. 20, 2019 is incorporated herein by reference in its entirety.

### BACKGROUND

### Technological Field

The present disclosure relates to a medical information processing apparatus and a computer readable storage medium.

### Description of the Related Art

In the medical field, technological innovation by deep learning has realized technologies for detecting multiple types of lesions at a time in a medical image. For example, there is a technology that can detect many types of lesions, such as nodule, mass, interstitial opacity, consolidation, bronchus inflammation and hyperinflation, at a time in a medical image of chest, using deep learning.

Lesion-detected regions are often expressed by being superimposed on images in the form of heatmaps, rectangles or the like. When lesion-detected regions of many types of lesions are displayed at a time, more marks are displayed as compared with a conventional case where a lesion-detected region(s) of one type of lesion is displayed. This makes it difficult for a doctor(s) to understand which mark(s) is an important indication. Lesion-detected regions of many types of lesions may be output by being superimposed one by one on an image(s), but it is inefficient to display all the lesion-detected regions and leave the judgment to the doctor. Further, it is undesirable to send all the heatmaps to an interpretation terminal of a PACS (Picture Archiving and Communication System) because this strains its storage capacity.

There is disclosed, for example, in JP 2013-517914 A prioritizing images obtained by an examination system, such as an imaging modality.

### SUMMARY

However, the technology disclosed in JP 2013-517914 A is a technology of prioritizing images by comparing the images with other images, and does not allow an interpreter(s) to recognize a lesion-detected region(s) that is important in interpretation, when two or more lesions are detected in an image of a subject site (part).

Objects of the present disclosure include allowing an interpreter(s) to easily recognize a lesion-detected region(s) that is important in interpretation, when two or more lesions are detected in a medical image of a subject site.

In order to achieve at least one of the objects, according to an aspect of the present disclosure, there is provided a medical information processing apparatus including a hardware processor that:

determines, based on a predetermined condition, priority degrees of lesion-detected regions of multiple types of lesions detected in a medical image; and

makes display forms of the lesion-detected regions detected in the medical image differ according to the determined priority degrees.

In order to achieve at least one of the objects, according to another aspect of the present disclosure, there is provided a non-transitory computer readable storage medium storing a program to cause a computer to:

determine, based on a predetermined condition, priority degrees of lesion-detected regions of multiple types of lesions detected in a medical image; and

make display forms of the lesion-detected regions detected in the medical image differ according to the determined priority degrees.

### BRIEF DESCRIPTION OF THE DRAWINGS

The advantages and features provided by one or more embodiments of the present invention will become more fully understood from the detailed description given hereinbelow and the appended drawings that are given by way of illustration only, and thus are not intended as a definition of the limits of the present invention, wherein:

FIG. 1 shows an overall configuration of a medical image display system according to an embodiment(s) of the present disclosure;

FIG. 2 is a block diagram showing a functional configuration of a medical information processing apparatus shown in FIG. 1;

FIG. 3 shows an example of a priority degree determination table;

FIG. 4 shows an example of a processing method table;

FIG. 5 is a flowchart of detection result information processing that is performed by a controller shown in FIG. 1;

FIG. 6 is an illustration for explaining a method of identifying a lesion-detected region based on heatmap information;

FIG. 7 is an illustration for explaining a method of calculating a gradient of certainty degrees of a lesion;

FIG. 8A shows an example of an image in which heatmap information of multiple types of lesions detected in a present medical image is colored, and superimposed and displayed as it is on the present medical image;

FIG. 8B shows an example of an image in which heatmap information of multiple types of lesions detected in a past medical image is colored, and superimposed and displayed as it is on the past medical image;

FIG. 9A shows an example of an image in which display information of the detection results (lesion-detected regions) obtained by the detection result information processing is superimposed and displayed on the present medical image shown in FIG. 8A;

FIG. 9B shows another example of the image in which the display information of the detection results (lesion-detected regions) obtained by the detection result information processing is superimposed and displayed on the present medical image shown in FIG. 8A;

FIG. 9C shows another example of the image in which the display information of the detection results (lesion-detected regions) obtained by the detection result information processing is superimposed and displayed on the present medical image shown in FIG. 8A; and

FIG. 10 is an illustration for explaining another use of the detection result information.

### DETAILED DESCRIPTION OF EMBODIMENTS

Hereinafter, one or more embodiments of the present invention will be described with reference to the drawings. However, the scope of the present invention is not limited to the disclosed embodiments or illustrated examples.

### [Configuration of Medical Image Display System 100]

First, configuration of an embodiment(s) will be described.

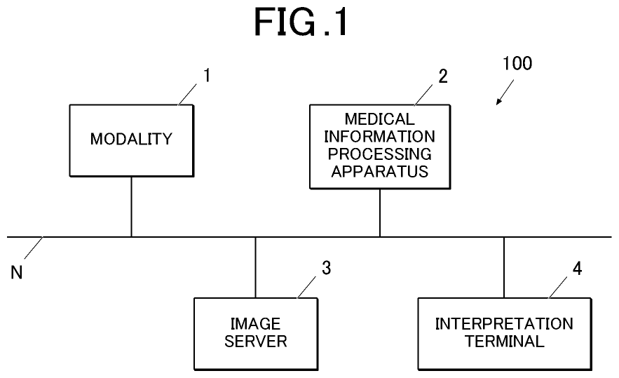

FIG. 1 shows an overall configuration of a medical image display system 100 according to an embodiment(s).

As shown in FIG. 1, the medical image display system 100 is configured such that a modality 1, a medical information processing apparatus 2, an image server 3 and an interpretation terminal 4 are connected to one another via a communication network N, such as a LAN (Local Area Network), a WAN (Wide Area Network) and/or Internet. The apparatuses constituting the medical image display system 100 are in conformity with HL7 (Health Level Seven) standard and DICOM standard, and communicate with one another in accordance with the HL7 standard and the DICOM standard.

The modality 1 is an image generating apparatus, such as an X-ray imaging apparatus (DR, CR), an ultrasonic diagnostic apparatus (US), a CT or an MRI, and generates a medical image(s) by photographing, as a subject, a site of a patient to be examined on the basis of examination order information sent from, for example, an RIS (Radiology Information System) (not shown). In accordance with the DICOM standard, the modality 1 writes supplementary information (patient information, examination information, image ID, slice numbers, etc.) in the header of an image file of the medical image, thereby attaching the supplementary information to the medical image, and sends the medical image with the supplementary information attached to the medical information processing apparatus 2 and the image server 3.

The medical information processing apparatus 2 detects multiple types of lesions in the medical image generated by the modality 1, determines priority degrees of regions of the detected lesions (lesion-detected regions), and generates display information of the lesion-detected regions by processing the detection result information such that their display forms differ according to their determined priority degrees. The medical information processing apparatus 2 is a PC, a portable terminal or a dedicated apparatus.

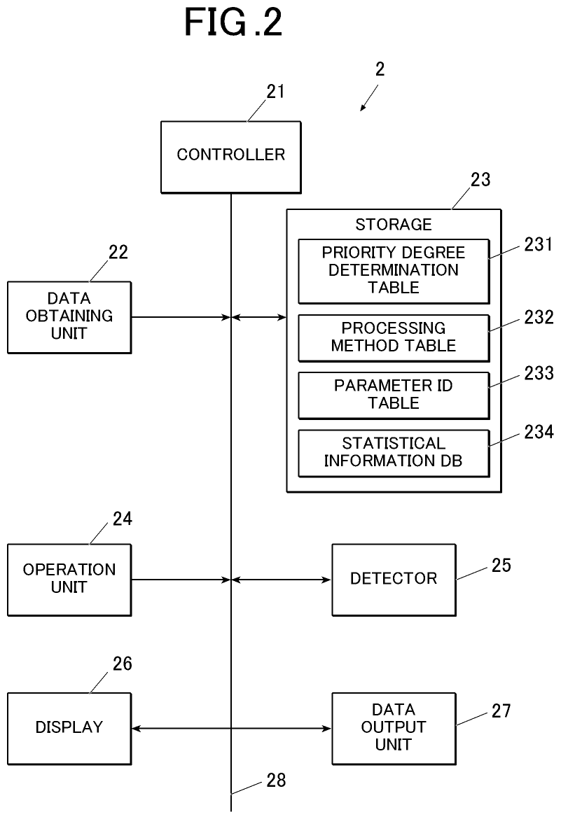

FIG. 2 is a block diagram showing a functional configuration of the medical information processing apparatus 2. As shown in FIG. 2, the medical information processing apparatus 2 includes a controller 21 (hardware processor), a data obtaining unit 22, a storage 23, an operation unit 24, a detector 25, a display 26 and a data output unit 27, and these components are connected to one another via a bus 28.

The controller 21 includes a CPU (Central Processing Unit) and a RAM (Random Access Memory), and comprehensively controls operation of each component of the medical information processing apparatus 2. The controller 21 reads out various programs stored in the storage 23, loads the read programs into the RAM, and performs various types of processing including detection result information processing, which will be described later, in accordance with the loaded programs.

The data obtaining unit 22 is for obtaining, from an external apparatus(es), image data of a medical image(s) and/or the detection result information on lesions detected in the medical image. The data obtaining unit 22 is constituted by a network interface or the like, and receives data from an external apparatus(es) connected via the communication network N with a cable or wirelessly. Although the data obtaining unit 22 is constituted by a network interface or the like in this embodiment, it may be constituted by a port or the like into which a USB memory, an SD card or the like can be inserted.

The storage 23 is constituted by an HDD (Hard Disk Drive), a semiconductor memory and/or the like, and stores programs for performing various types of processing including the detection result information processing, which will be described later, and parameters, files and so forth necessary for executing the programs, for example.

The storage 23 stores, for example, a priority degree determination table 231, a processing method table 232, a parameter ID table 233 and a statistical information DB (DataBase) 234.

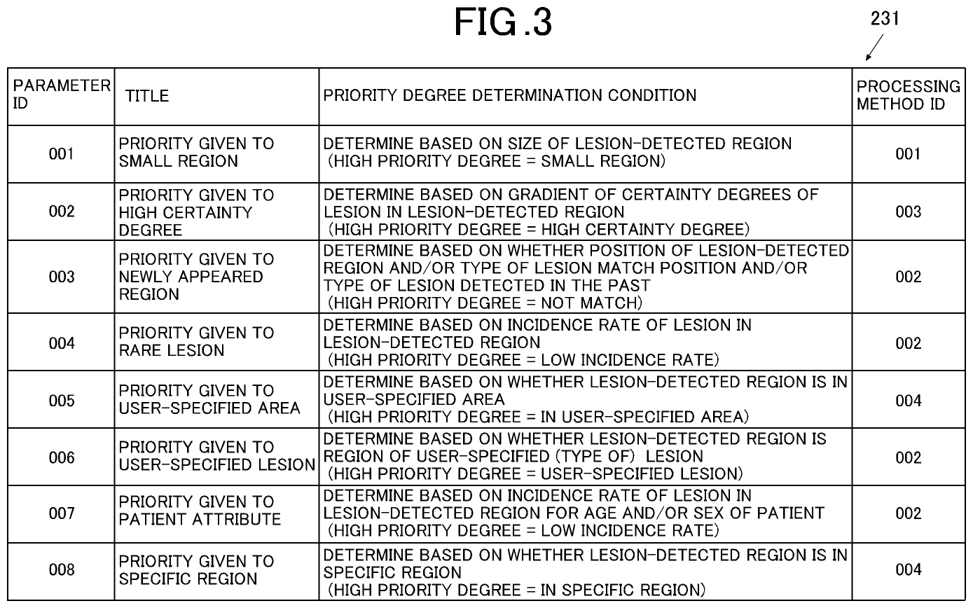

FIG. 3 shows an example of how data are stored in the priority degree determination table 231. As shown in FIG. 3, the priority degree determination table 231 has a “Parameter ID” field, a “Title” field, a “Priority Degree Determination Condition” field and a “Processing Method ID” field.

The “Parameter ID” field stores parameter IDs for identifying respective priority degree determination conditions each of which can be used for determining priority degrees of lesion-detected regions. The “Title” field stores titles of the respective priority degree determination conditions. The “Priority Degree Determination Condition” field stores details of the respective priority degree determination conditions. The “Processing Method ID” field stores processing method IDs for identifying respective processing methods each of which can be used for processing the detection result information according to the determined priority degrees.

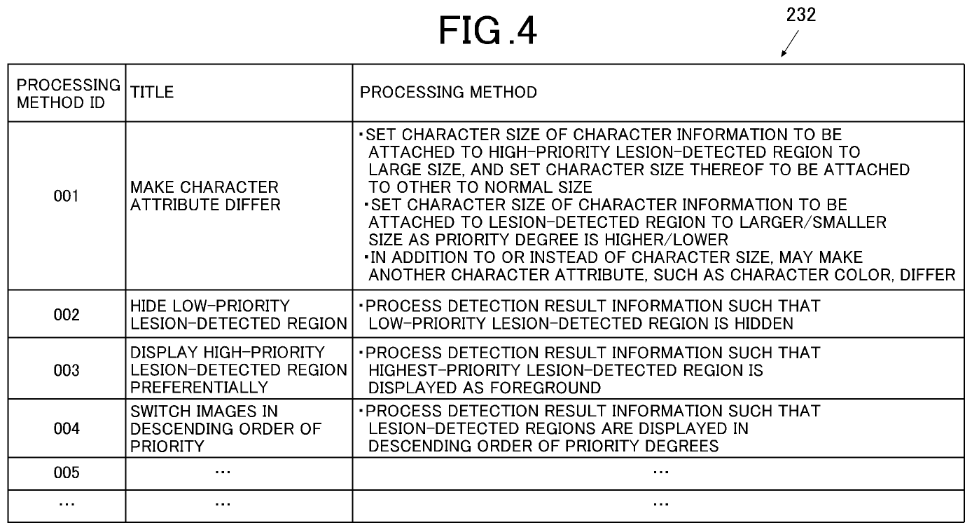

FIG. 4 shows an example of how data are stored in the processing method table 232. As shown in FIG. 4, the processing method table 232 has a “Processing Method ID” field, a “Title” field and a “Processing Method” field.

The “Processing Method ID” field stores the processing method IDs for identifying the respective processing methods each of which can be used for processing the detection result information on the basis of the priority degrees of the lesion-detected regions. The “Title” field stores titles of the respective processing methods. The “Processing Method” field stores details of the respective processing methods.

The parameter ID table 233 stores the parameter IDs in association with, for example, consultation departments that patients have consulted, user IDs, client departments that have made requests for interpretation, or examination purposes.

The parameter IDs can each be specified, for example, by a user(s) through the operation unit 24. For example, the user can specify a desired parameter ID, for example, by checking a checkbox for the desired parameter ID in a parameter ID specifying screen displayed on the display 26 with a predetermined operation. Alternatively, in order for the user to easily understand contents of the priority degree determination conditions, their titles or the like corresponding to the respective parameter IDs may be displayed, so that the user can specify a title. Allowing the user to specify the parameter ID(s) allows the user to freely set a condition(s) of lesion-detected regions to be displayed preferentially and to freely set their display forms according to their priority degrees. Alternatively, the parameter IDs may be set in advance by hard coding.

For each consultation department, user ID, client department or examination purpose, only one parameter ID may be specified/set, or two or more parameter IDs may be specified/set with the order of priority.

Specifying/setting one or more parameter IDs for each consultation department enables determination of the priority degrees peculiar to each consultation department. Specifying/setting one or more parameter IDs for each user enables determination of the priority degrees desired by each user. Client departments are each information for a radiologist(s), who interprets images, to identify which consultation department has made a request for interpretation, and specifying/setting one or more parameter IDs for each client department enables determination of the priority degrees peculiar to each department that a patient has consulted. Examination purposes are each information indicating a purpose of an examination, such as a cancer examination, an examination on an outpatient or a follow-up on a hospitalized patient, and specifying/setting one or more parameter IDs for each examination purpose makes it possible to change how to determine the priority degrees in accordance with an examination purpose (e.g. in the case of a follow-up on a hospitalized patient, priority is given to newly appeared regions (parameter ID=003)).

The statistical information DB 234 is a database that stores statistical information, such as incidence rates of lesions by age and sex.

The operation unit 24 includes a keyboard including various keys, a pointing device, such as a mouse, and/or a touchscreen attached to the display 26, and can be operated by the user. The operation unit 24 outputs, to the controller 21, input operation signals corresponding to key operations on the keyboard, mouse operations or positions of touch operations on the touchscreen.

A portable terminal may be connected to the medical information processing apparatus 2 with a cable or wirelessly, and a touchscreen and/or buttons on a liquid crystal display panel of the portable terminal may be used as the operation unit 24.

The detector 25 detects multiple types of lesions in a medical image(s) obtained by the data obtaining unit 22, and outputs the detection result information on the multiple types of lesions.

In this embodiment, the detector 25 detects multiple types of lesions in an input medical image by using machine learning models created by such as deep learning of a large amount of training data (pairs each of which is constituted by a medical image showing a lesion and a correct label (lesion region in the medical image, lesion/disease (lesion type) name, etc.), and associates and outputs the detection result information with the medical image to the controller 21.

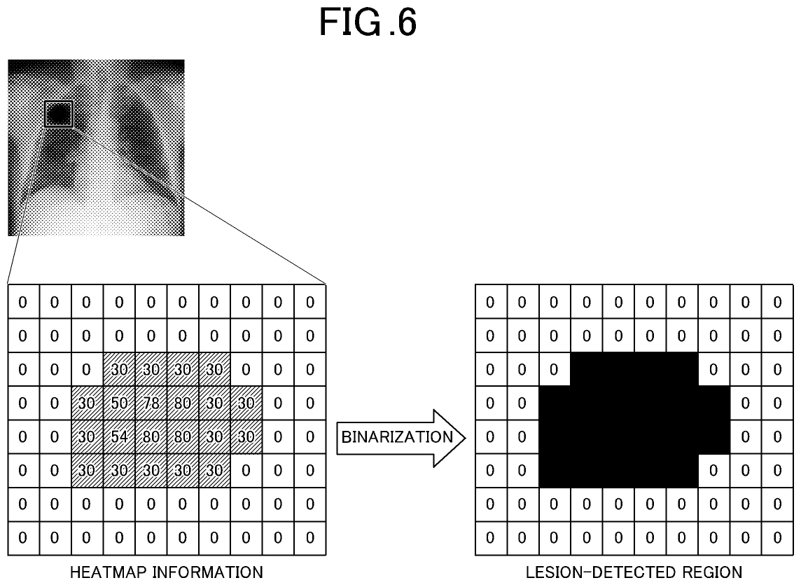

The detection result information is output for each lesion type. The detection result information includes: heatmap information (shown in FIG. 6) indicating certainty degrees of a lesion in respective pixels of a medical image; and the supplementary information (lesion type, image ID for identifying the medical image, examination ID, etc.). A certainty degree of 0 indicates no possibility of a lesion, and a higher certainty degree indicates a higher possibility of a lesion.

The display 26 includes a monitor, such as an LCD (Liquid Crystal Display), and displays various screens in accordance with commands of display signals input from the controller 21. The number of monitors may be one or more than one.

The data output unit 27 is for outputting information processed by the medical information processing apparatus 2 to the outside. Examples of the data output unit 27 include: a network interface for communicating with other systems (image server 3, etc.); connectors for connecting with external apparatuses (display apparatus, printer, etc.); and ports for various media (USB memory, etc.).

The image server 3 is, for example, a server of a PACS (Picture Archiving and Communication System), and associates and stores, in a database, each medical image output from the modality 1 with the patient information (patient ID, name, birth date, age, sex, height, weight, etc.), the examination information (examination ID, examination date and time, modality type, examination site, client department, examination purpose, etc.), the image ID of the medical image, and the detection result information and the display information of lesion-detected regions output from the medical information processing apparatus 2.

The image server 3 reads out, from the database, a medical image and the display information of lesion-detected regions associated with the medical image, which have been requested by the interpretation terminal 4, and causes the interpretation terminal 4 to display these.

The interpretation terminal 4 is a computer apparatus that includes a controller, an operation unit, a display, a storage and a communication unit, and reads out a medical image and its display information of lesion-detected regions from the image server 3 by making a request to the image server 3, and displays these for interpretation.

### [Operation of Medical Information Processing Apparatus 2]

Next, operation of the medical information processing apparatus 2 will be described.

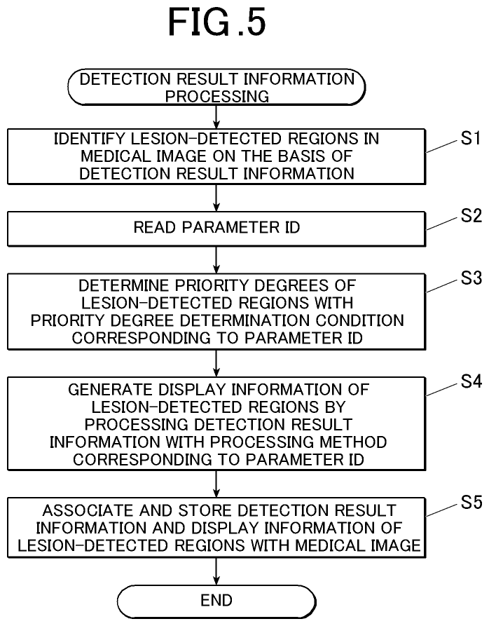

FIG. 5 is a flowchart of the detection result information processing that is performed on the detection result information obtained by the detector 25 detecting multiple types of lesions in a medical image input from the modality 1 or on the detection result information obtained by the data obtaining unit 22 from an external apparatus (detection result information obtained by an external apparatus detecting multiple types of lesions in a medical image). The detection result information processing is performed by the controller 21 and the program(s) stored in the storage 23 in cooperation with one another.

First, the controller 21 identifies lesion-detected regions in a medical image on the basis of the detection result information (Step S1).

For example, as shown in FIG. 6, the controller 21 binarizes the heatmap information by using a predetermined threshold value, and identifies a region(s) equal to or larger than the threshold value (region filled with black in FIG. 6) as a lesion-detected region(s).

Next, the controller 21 reads a parameter ID from the parameter ID table 233 in the storage 23 (Step S2).

When the parameter IDs are stored for respective users in the parameter ID table 233, the controller 21 reads a parameter ID stored in association with the user ID of a user who currently logs in to the medical information processing apparatus 2.

When the parameter IDs are stored for respective consultation departments in the parameter ID table 233, the controller 21 reads a parameter ID stored in association with the consultation department to which the logged-in user belongs. Information indicating which user belongs to which consultation department is stored in the storage 23.

When the parameter IDs are stored for respective client departments in the parameter ID table 233, the controller 21 reads a parameter ID stored in association with the client department included in the DICOM header (supplementary information for the medical image) or the examination order information.

When the parameter IDs are stored for respective examination purposes in the parameter ID table 233, the controller 21 reads a parameter ID stored in association with the examination purpose included in the DICOM header or the examination order information.

Next, the controller 21 reads the priority degree determination table 231, reads out a priority degree determination condition corresponding to the parameter ID read in Step S2, and determines priority degrees of the lesion-detected regions with the read priority degree determination condition (Step S3).

When the read parameter ID is 001 (priority given to small region), the controller 21 obtains information on the size of each lesion-detected region (area, volume, length of the longer axis, etc.) identified in Step S1, and determines the priority degree thereof on the basis of the obtained size. More specifically, the controller 21 determines lesion-detected regions having a small size (e.g. area, volume, length of the longer axis, etc.) as high priority (having a high priority degree). The controller 21 may determine, on the basis of whether the size of each lesion-detected region is smaller than each of one or more preset threshold values, which priority degree (level) of multiple levels of priority the lesion-detected region has (belongs to), or may determine the priority degrees of the respective lesion-detected regions by assigning numbers to the respective lesion-detected regions in ascending order of size and determining the numbers as the priority degrees (the smaller the number is, the higher the priority degree is).

The area of each lesion-detected region can be obtained from the number of pixels of the lesion-detected region, for example. The length (dimension) in the longer axis direction of each lesion-detected region can be obtained from the number of pixels of the maximum width of the lesion-detected region, for example.

Giving priority to small regions makes it possible to give priority to small lesion-detected regions that are prone to be overlooked.

When the read parameter ID is 002 (priority given to high certainty degree), the controller 21 determines the priority degree of each lesion-detected region on the basis of the gradient of certainty degrees of the lesion in the lesion-detected region. More specifically, the controller 21 determines lesion-detected regions having a large gradient of certainty degrees of a lesion as high priority (having a high priority degree). The controller 21 may determine, on the basis of whether the gradient of certainty degrees of the lesion in each lesion-detected region is larger than each of one or more preset threshold values, which priority degree (level) of multiple levels of priority the lesion-detected region has (belongs to), or may determine the priority degrees of the respective lesion-detected regions by assigning numbers to the respective lesion-detected regions in descending order of gradient of certainty degrees and determining the numbers as the priority degrees (the smaller the number is, the higher the priority degree is).

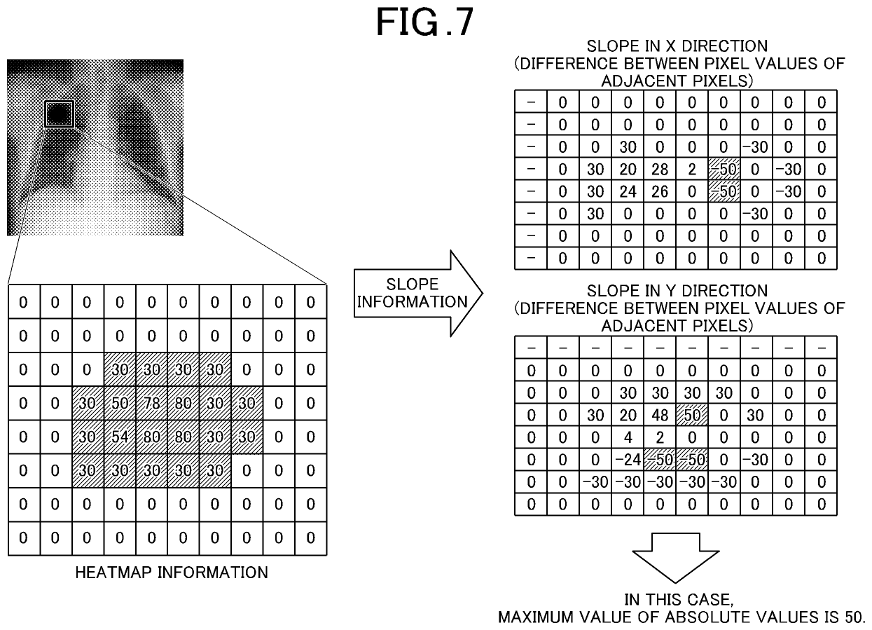

The gradient of certainty degrees of the lesion in each lesion-detected region can be obtained, for example, as shown in FIG. 7, by calculating slopes in the x direction (differences between pixel values of pixels adjacent to one another in the x direction) and slopes in the y direction (differences between pixel values of pixels adjacent to one another in the y direction) of the heatmap information, and regarding the maximum value (“50” in FIG. 7) of the absolute values of the slopes as a representative value of the slopes of certainty degrees. Then, the controller 21 determines a lesion-detected region having a larger representative value of the slopes of certainty degrees (having a steeper slope of certainty degrees) as higher priority (having a higher priority degree).

Giving priority to high (gradients of) certainty degrees makes it possible to give priority to lesion-detected regions having high (gradients of) certainty degrees.

When the read parameter ID is 003 (priority given to newly appeared region), the controller 21 determines the priority degree of each lesion-detected region on the basis of whether the position of the lesion-detected region and/or the type of the lesion therein match the position and/or the type of a lesion(s) detected in the past from the subject of the medical image. More specifically, the controller 21 obtains an examination result (interpretation report, detection result information, etc.) in the past about the same patient (subject) from the image server 3, compares the type and/or the position information of each lesion detected by the detector 25 or the like in the present medical image and included in the detection result information (coordinate information of each region extracted by binarizing the heatmap information by using a predetermined threshold) with the type and/or the position information (coordinate information) of a lesion(s) detected in the past examination, and determines lesion-detected regions having the comparison result of matching as low priority and lesion-detected regions having the comparison result of not matching as high priority (two levels of priority).

Giving priority to newly appeared regions makes it possible to give priority to newly detected regions (new lesion-detected regions).

When the read parameter ID is 004 (priority given to rare lesion), the controller 21 obtains the incidence rate of each (type of) lesion detected in the target medical image from the statistical information stored in the statistical information DB 234, and determines the priority degree thereof on the basis of the obtained incidence rate. More specifically, the controller 21 determines lesion-detected regions of lesions having a low incidence rate as high priority (having a high priority degree). For example, the controller 21 determines lesion-detected regions of lesions having an incidence rate smaller (lower) than a preset threshold value as high priority and lesion-detected regions of lesions having an incidence rate larger (higher) than the preset threshold value as low priority.

Giving priority to rare lesions makes it possible to give priority to lesions that rarely appear (that are unfamiliar to the doctor).

When the read parameter ID is 005 (priority given to user-specified area), the controller 21 determines the priority degree of each lesion-detected region on the basis of whether the lesion-detected region is in an area(s) specified by the user (e.g. doctor in charge). More specifically, the controller 21 compares the position information of each lesion-detected region (coordinate information of each region extracted by binarizing the heatmap information by using a predetermined threshold value) with a user-specified area(s), which has been specified through the operation unit 24, on the medial image displayed on the display 26, and determines lesion-detected regions located in the user-specified area as high priority and lesion-detected regions located outside the user-specified area as low priority.

Giving priority to a user-specified area(s) makes it possible to set, to high priority, lesion-detected regions located in the area to which the user would like an interpreter to pay attention. The controller 21 may determine lesion-detected regions located outside a user-specified area as high priority and lesion-detected regions located in the user-specified area as low priority. Determining lesion-detected regions located outside a user-specified area as high priority makes it possible to call attention to the lesion-detected regions that are in an area to which the user has not paid much attention.

The user may specify an area, for example, for each examination, or may specify an area in advance so that the area is stored in the storage 23 in advance.

When the read parameter ID is 006 (priority given to user-specified lesion), the controller 21 determines the priority degree of each lesion-detected region on the basis of whether the lesion-detected region is a lesion-detected region of a lesion of a type specified by the user. More specifically, the controller 21 determines whether the type of the lesion in each lesion-detected region matches a lesion type(s) specified by the user from a predetermined list through the operation unit 24, and determines lesion-detected regions having the determination result of matching as high priority and lesion-detected regions having the determination result of not matching as low priority.

Giving priority to user-specified lesions makes it possible to give priority to lesion types to which the user pays special attention.

The user may specify a (type(s) of) lesion(s), for example, for each examination, or may specify a (type(s) of) lesion for each site in advance so that the lesion for each site is stored in the storage 23 in advance.

When the read parameter ID is 007 (priority given to patient attribute), the controller 21 obtains the incidence rate of each lesion detected in the target medical image for the age and/or the sex of the patient from the statistical information stored in the statistical information DB 234, and determines the priority degree thereof on the basis of the obtained incidence rate. For example, the controller 21 determines lesion-detected regions of lesions having an incidence rate lower (smaller) than a predetermined threshold value as high priority and lesion-detected regions of lesions having an incidence rate equal to or larger (higher) than the predetermined threshold value as low priority.

Giving priority to patient attributes makes it possible to give priority to lesions that rarely appear (that the doctor may overlook) in the age and/or the sex of a patient.

When the read parameter ID is 008 (priority given to specific region), the controller 21 determines the priority degree of each lesion-detected region on the basis of whether the lesion-detected region is in a specific region(s) set by default. More specifically, the controller 21 determines lesion-detected regions located in a specific region(s) as high priority and lesion-detected regions located outside the specific region as low priority.

Giving priority to a specific region(s) makes it possible to give priority to lesions in the specific region.

When two or more parameter IDs are stored with the order of priority in the parameter ID table 233, the controller 21 reads out a priority degree determination condition corresponding to the highest parameter ID in the order of priority, and determines the priority degrees therewith. When lesion-detected regions having the same priority degree are present as a result of the above determination, the controller 21 may read out a priority degree determination condition corresponding to the second-highest parameter ID in the order of priority, and determine the priority degrees therewith.

Next, the controller 21 reads the processing method table 232, reads out a processing method having a processing method ID corresponding to the priority degree determination condition in the priority degree determination table 231, the priority degree determination condition having been used for determining the priority degrees, and generates the display information of the lesion-detected regions by processing the detection result information with the read processing method (Step S4).

In Step S4, by processing the detection result information, the controller 21 generates the display information of the lesion-detected regions (heatmap display information of each lesion-detected region and character information indicating the type of the lesion in each lesion-detected region) that is superimposed on the medical image. The heatmap display information is, for example, information colored according to the values of the certainty degrees.

Examples of the processing methods corresponding to the respective processing method IDs include the following.

(1) Make attribute of characters (character attribute) differ (processing method ID=001).
Set character size of character information (lesion type, etc.) on a high-priority lesion-detected region(s) to a large size, and set character size thereof on the other(s) to a normal size.
Set character size of character information on each lesion-detected region to be larger/smaller as the priority degree is higher/lower.
In addition to or instead of character size, may make another character attribute, such as character color, differ.

High-priority lesion-detected regions are lesion-detected regions having a priority degree equal to or higher than a preset reference priority degree, whereas low-priority lesion-detected regions are lesion-detected regions having a priority degree lower than the preset reference priority degree.

(2) Hide low-priority lesion-detected region (processing method ID=002).
Generate the display information of lesion-detected regions by processing the detection result information (heatmap information) such that low-priority lesion-detected regions are hidden.
   (3) Display high-priority lesion-detected region preferentially (processing method ID=003).
Generate the display information of lesion-detected regions by processing the detection result information (heatmap information) such that the lesion-detected regions having higher priority degrees are displayed more forward.
   (4) Switch lesion-detected regions (images) to display in descending order of priority (processing method ID=004).
Generate the display information of lesion-detected regions by processing the detection result information (heatmap information) such that the lesion-detected regions are successively output in descending order of their priority degrees.

That is, in Step S4, the controller 21 generates the display information of the lesion-detected regions, which is superimposed on the medical image, such that their display forms differ according to their priority degrees. Consequently, when the lesion-detected regions are superimposed and displayed on the medical image, the display forms of the lesion-detected regions can be different from one another according to their priority degrees.

Next, the controller 21 associates and stores the detection result information and the display information of the lesion-detected regions with the medical image (Step S5), and ends the detection result information processing.

For example, the controller 21 sends the medical image and the display information of the lesion-detected regions to the image server 3 by using the data output unit 27, thereby storing the medical image, the detection result information and the display information of the lesion-detected regions in association with one another in the database of the image server 3.

Alternatively, the controller 21 stores the medical image, the detection result information and the display information of the lesion-detected regions in association with one another in the storage 23.

The medical image stored in the database of the image server 3 is displayed on a display (not shown) of the interpretation terminal 4 in response to a request from the interpretation terminal 4. At the time, the display information of the lesion-detected regions is displayed on the medical image by being superimposed thereon. Alternatively, the medical image stored in the storage 23 is displayed on the display 26 in response to an operation made through the operation unit 24. At the time, the display information of the lesion-detected regions is displayed on the medical image by being superimposed thereon.

The display 26 may include a color monitor and a monochrome monitor. Monochrome monitors can perform display with higher brightness and contrast than color monitors. When the display 26 has a color monitor and a monochrome monitor, medical images are displayed on the monochrome monitor by default. However, the display information of lesion-detected regions is in color, and hence when displayed on the monochrome monitor, the lesion-detected regions are difficult to recognize. It is therefore preferable that when the display information of lesion-detected regions is superimposed on a medical image, the controller 21 display the medical image on the monochrome monitor by default, but can display the medical image on the color monitor in response to a predetermined operation. For example, when a medical image displayed on the monochrome monitor is clicked through the operation unit 24, the controller 21 displays, on the color monitor, the medical image on which the display information of lesion-detected regions is superimposed. Alternatively, when a medical image on which the display information of lesion-detected regions is superimposed is displayed on the monochrome monitor, the controller 21 outputs a warning indicating that the medical image includes a color item(s) and is not properly displayed. For example, the controller 21 displays, on or near the displayed medical image, an icon indicating that the medical image includes color information and is not properly displayed, and when the icon is clicked through the operation unit 24, the controller 21 displays, on the color monitor, the medical image on which the display information of lesion-detected regions is superimposed. It is preferable that the same display control be performed when a medical image on which the display information of lesion-detected regions is superimposed is displayed on the display of the interpretation terminal 4.

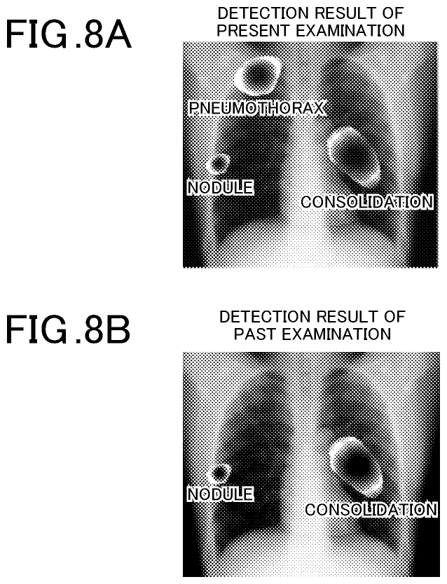

FIG. 8A shows an example of an image in which the heatmap information of multiple types of lesions detected in a present medical image of a patient is colored, and superimposed and displayed as it is on the present medical image. FIG. 8B shows an example of an image in which the heatmap information of multiple types of lesions detected in a past medical image is colored, and superimposed and displayed as it is on the past medical image.

In FIG. 8A, the detection result information on each of the multiple types of lesions is displayed in the same display form, and hence it is difficult to understand which lesion-detected region(s) is important.

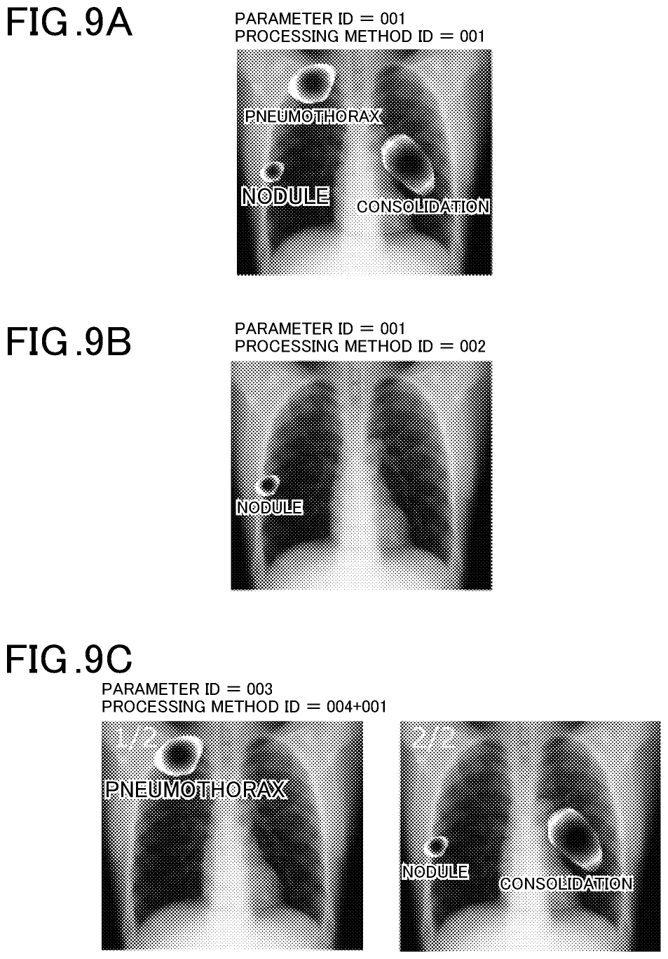

FIG. 9A to FIG. 9C show examples of an image in which the display information of detection results (lesion-detected regions) obtained by the detection result information processing is superimposed and displayed on the present medical image shown in FIG. 8A.

FIG. 9A shows an image in the case where the priority degrees have been determined with the parameter ID of 001 (priority given to small region) and the detection result information has been processed with the processing method ID of 001 (make character attribute differ). That is, characters of a nodule determined as high priority by giving priority to small regions are displayed with larger characters than those of the other lesions. This can display, with emphasis, small lesion-detected regions that are hard for an interpreter to notice.

FIG. 9B shows an image in the case where the priority degrees have been determined with the parameter ID of 001 (priority given to small region) and the detection result information has been processed with the processing method ID of 002 (hide low-priority lesion-detected region). That is, lesions determined as low priority by giving priority to small regions are hidden (not displayed), and only the nodule determined as high priority is displayed. This can display only small lesion-detected regions that are hard for an interpreter to notice, and consequently allows an interpreter to interpret small lesion-detected regions with focus thereon.

FIG. 9C shows an image in the case where the priority degrees have been determined with the parameter ID of 003 (priority given to newly appeared region) and the detection result information has been processed with the processing method IDs of 004 and 001 (switch images in descending order of priority+make character attribute differ). That is, a pneumothorax-detected region that is not present in the past medical image is first displayed with large characters, and thereafter it is switched and lesion-detected regions of the other lesions are displayed with small characters. This allows an interpreter to first interpret new lesion-detected regions, which are not present in a past image(s), with focus thereon and also check pre-existing lesions.

Thus, the detection result information processing processes the detection result information on multiple types of lesions according to the priority degrees, which have been determined on the basis of a predetermined priority degree determination condition(s), thereby making the display forms of the lesion-detected regions differ according to the priority degrees, and consequently allows an interpreter to easily recognize lesion-detected regions that are important in interpretation.

### [Another Use of Detection Result Information]

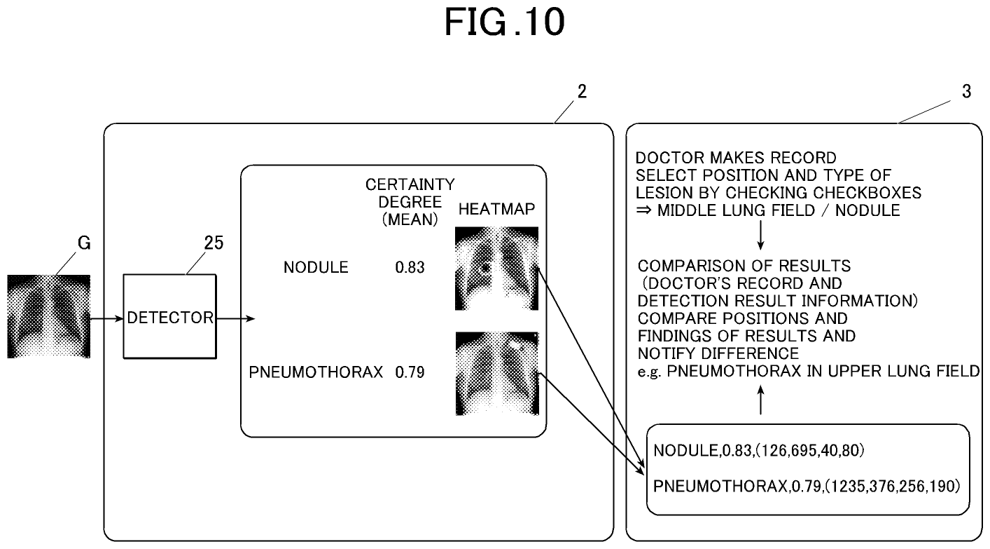

As another use of the detection result information, the PACS (image server 3+interpretation terminal 4) may compare the detection result information with a position(s) of a lesion(s) recorded by a doctor and notify their difference. FIG. 10 shows data and procedure in this case.

As shown in FIG. 10, when a medical image G is input to the medical information processing apparatus 2, the detector 25 detects multiple types of lesions in the medical image G and outputs the detection result information. As described above, the detection result information includes, for each lesion type, the heatmap information and the supplementary information including a lesion type. The controller 21, for each lesion type, calculates the mean of certainty degrees from the heatmap information, attaches the calculated mean to the supplementary information, and outputs these to the image server 3.

When a doctor selects a position and a finding (type) of a lesion through the interpretation terminal 4, the image server 3, with the CPU and the program working in cooperation with one another, compares the position of each lesion-detected region and the type of the lesion therein included in the detection result information input from the medical information processing apparatus 2 with the position and the type of the lesion selected by the doctor.

The doctor may select the position of the lesion by specifying a region of the lesion on the medical image through a mouse or the like or by using a checking method. The checking method is, in the case of chest, selecting one from the upper lung field, the middle lung field and the lower lung field into which a lung field is divided in advance, thereby selecting a region where a lesion is located. FIG. 10 shows that the middle lung field and nodule have been selected by using the checking method.

For example, the image server 3 first calculates coordinates (x, y, h (height), w (width)) of a representative point of each lesion-detected region included in the detection result information. The representative point may be the centroid of a lesion-detected region, a point where the certainty degree is the maximum value, or the centroid of a region where the certainty degree(s) is a predetermined value or larger. Next, the image server 3 determines whether the type of each lesion detected by the detector 25 has been specified by the doctor as a finding, and if so, determines whether the calculated representative point is included in the position specified by the doctor. When there is a lesion of a type not specified by the doctor, or when there is a lesion of a type specified by the doctor but its representative point is not included in the position specified by the doctor, the image server 3 notifies the lesion type to the doctor by, for example, causing the interpretation terminal 4 to display the lesion type. For example, in FIG. 10, the detection result information includes pneumothorax in the upper lung field, but pneumothorax is not included in the doctor's record. Hence, the image server 3 causes the interpretation terminal 4 to display, for example, “Pneumothorax in Upper Lung Field”.

When notifying the difference, the image server 3 may causes the interpretation terminal 4 to display the detection result information too output from the medical information processing apparatus 2. For example, the image server 3 may cause the interpretation terminal 4 to display the detection result information (heatmaps) so as to be superimposed on the medical image and also display the certainty degrees of the respective (types of) lesions at a corner of the screen. When there is a lesion-detected region that is the same in type and position as a lesion detected in a past examination, an “Identical” mark or the like may be attached to the lesion-detected region, or the lesion-detected region may be hidden. Further, the reason(s) why “Pneumothorax in Upper Lung Field” is notified (in the case shown in FIG. 10, it is because pneumothorax in the upper lung field is included in the detection result information output from the medical information processing apparatus 2 but not specified by the doctor) may be displayed. To notify “Pneumothorax in Upper Lung Field” or the like, characters, a heatmap or both may be output.

As described above, the controller 21 of the medical information processing apparatus 2 determines, on the basis of a predetermined condition, the priority degrees of lesion-detected regions of multiple types of lesions detected in a medical image, and generates the display information of the lesion-detected regions such that the display forms of the lesion-detected regions detected in the medical image differ according to the determined priority degrees.

This allows an interpreter to easily recognize lesion-detected regions that are important in interpretation, and as a result, enables efficient interpretation.

The description of the embodiment above is merely one of preferable examples of the medical image display system, the medical information processing apparatus or the like of the present disclosure, and hence is not intended to limit the present invention.

For example, in the above embodiment, the supplementary information included in the detection result information includes the character strings expressing the respective lesion types. However, the supplementary information may include lesion codes identifying the respective lesion types. Then, the medical information processing apparatus 2 and the image server 3 may store the character strings, which expressing the respective lesion types, corresponding to the respective lesion codes, and display the character strings, thereby displaying the lesion types.

Further, for example, in the above embodiment, the digital data represents the heatmap information of one type of lesion. However, each bit in a bit string of each pixel may be made to have meaning, and one digital data may represent the heatmap information of multiple types of lesions. For example, in the case of digital data in which each pixel is composed of 16 bits, lesion types may be assigned to the bits such that the first 4 bits represent the heatmap information of nodule, the next 4 bits represent the heatmap information of pneumothorax, and so forth, so that the digital data represents the heatmap information of multiple types of lesions. This can reduce the data amount of the heatmap information of multiple types of lesions.

Further, for example, in the above embodiment, the present invention is applied to the case where multiple types of lesions are detected in a medical image at a time by deep learning, and the lesion-detected regions in the medical image are displayed on the basis of the obtained detection result information. However, multiple types of lesions may not be detected by machine learning. For example, the present invention may be applied to a case where multiple types of lesions are detected in a medical image by using multiple types of software each of which is for detecting one type of lesion, and the lesion-detected regions in the medical image are displayed on the basis of the obtained detection result information.

Further, for example, in the above, a hard disk, a nonvolatile semiconductor memory or the like is used as a computer readable medium of the programs of the present disclosure. However, this is not a limitation. As the computer readable medium, a portable storage medium, such as a CD-ROM, can also be used. Further, as a medium to provide data of the programs of the present disclosure via a communication line, a carrier wave can be used.

Further, the detailed configuration and detailed operation of each component of the medical image display system 100 can also be appropriately modified without departing from the scope of the present invention.

Although one or more embodiments of the present invention have been described and illustrated in detail, the disclosed embodiments are made for purposes of not limitation but illustration and example only. The scope of the present invention should be interpreted by terms of the appended claims

What is claimed is:

1. A medical information processing apparatus comprising a hardware processor that:
determines, based on a predetermined condition, priority degrees of lesion-detected regions of multiple types of lesions detected in a medical image; and
makes display forms of the lesion-detected regions detected in the medical image differ according to the determined priority degrees.

2. The medical information processing apparatus according to claim 1, wherein the hardware processor determines the priority degrees of the lesion-detected regions based on whether the lesion-detected regions are each a lesion-detected region of a lesion of a type specified by a user.

3. The medical information processing apparatus according to claim 1, wherein the hardware processor determines the priority degrees of the lesion-detected regions based on whether the lesion-detected regions are each in an area specified by a user.

4. The medical information processing apparatus according to claim 1, wherein the hardware processor determines the priority degrees of the lesion-detected regions based on aspects of the lesion-detected regions.

5. The medical information processing apparatus according to claim 4, wherein the hardware processor determines the priority degrees of the lesion-detected regions based on sizes of the lesion-detected regions.

6. The medical information processing apparatus according to claim 4, wherein the hardware processor determines the priority degrees of the lesion-detected regions based on gradients of certainty degrees of the lesions in the lesion-detected regions.

7. The medical information processing apparatus according to claim 1, wherein the hardware processor determines the priority degrees of the lesion-detected regions based on whether positions of the lesion-detected regions and/or the types of the lesions each match a position and/or a type of a lesion previously detected from a subject of the medical image.

8. The medical information processing apparatus according to claim 1, wherein the hardware processor determines the priority degrees of the lesion-detected regions based on statistical information on the multiple types of the lesions.

9. The medical information processing apparatus according to claim 8, wherein the hardware processor determines the priority degrees of the lesion-detected regions based on incidence rates of the lesions in the lesion-detected regions obtained from the statistical information on the multiple types of the lesions.

10. The medical information processing apparatus according to claim 8, wherein the hardware processor determines the priority degrees of the lesion-detected regions based on incidence rates of the lesions in the lesion-detected regions for an age and/or a sex of a subject of the medical image obtained from the statistical information on the multiple types of the lesions.

11. The medical information processing apparatus according to claim 1, wherein the hardware processor determines the priority degrees of the lesion-detected regions based on whether the lesion-detected regions are each in a predetermined specific region.

12. The medical information processing apparatus according to claim 1, wherein the hardware processor generates display information of the lesion-detected regions such that sizes of character information on the lesion-detected regions differ according to the determined priority degrees.

13. The medical information processing apparatus according to claim 1, wherein the hardware processor generates display information of the lesion-detected regions such that, among the lesion-detected regions, a lesion-detected region the determined priority degree of which is equal to or higher than a preset reference is displayed, and a lesion-detected region the determined priority degree of which is lower than the preset reference is hidden.

14. The medical information processing apparatus according to claim 1, wherein the hardware processor generates display information of the lesion-detected regions such that the lesion-detected regions the determined priority degrees of which are higher are displayed more forward.

15. The medical information processing apparatus according to claim 1, wherein the hardware processor generates display information of the lesion-detected regions such that the lesion-detected regions are switched to be displayed in descending order of the determined priority degrees.

16. The medical information processing apparatus according to claim 1, further comprising an operation unit for a user to specify the condition based on which the priority degrees are determined.

17. A non-transitory computer readable storage medium storing a program to cause a computer to:
determine, based on a predetermined condition, priority degrees of lesion-detected regions of multiple types of lesions detected in a medical image; and
make display forms of the lesion-detected regions detected in the medical image differ according to the determined priority degrees.
